(19) United States
(12) Patent Application Publication
(10) Pub. No.: US 2023/0301973 A1
(43) Pub. Date: Sep. 28, 2023

(54) TREATMENT OF DIFFUSE INTRINSIC PONTINE GLIOMA

(71) Applicant: Jacob Ary Flohil, Delft (NL)

(72) Inventor: Jacob Ary Flohil, Delft (NL)

(21) Appl. No.: 16/468,168

(22) PCT Filed: Dec. 8, 2017
(86) PCT No.: PCT/NL2017/050826

§ 371 Date: Jun. 10, 2019

(30) Foreign Application Priority Data
Dec. 9, 2016 (NL) 2017972

(51) Int. Cl. A61K 31/4375; A61K 45/06; A61P 35/00; A61K 9/127

(52) U.S. Cl. CPC A61K 31/4375; A61K 9/127; A61K 45/06; A61P 35/00

(57) ABSTRACT

This disclosure relates to an inhibitor of MELK and/or ROR2, preferably for use in the treatment of diffuse intrinsic pontine glioma (DIPG), wherein the DIPG is preferably characterized by overexpression of MELK and/or overexpression of ROR2. The inhibitor may be combined with a P-glycoprotein inhibitor, an Abcb1a inhibitor, Abcb1b inhibitor, mannitol, or an Abcg2 inhibitor.

### CROSS-REFERENCE TO RELATED APPLICATIONS

This application is a national phase entry under 35 U.S.C. § 371 of International Patent Application PCT/NL2017/050826, filed Dec. 8, 2017, designating the United States of America and published in English as International Patent Publication WO 2018/106118 A1 on Jun. 14, 2018, which claims the benefit under Article 8 of the Patent Cooperation Treaty to Dutch Patent Application Serial No. 2017972, filed Dec. 9, 2016.

### TECHNICAL FIELD

The disclosure relates to an inhibitor of MELK and/or ROR2, preferably for use in the treatment of diffuse intrinsic pontine glioma (DIPG), wherein the DIPG is preferably characterized by overexpression of MELK and/or overexpression of ROR2.

### BACKGROUND

Diffuse intrinsic pontine glioma (DIPG) is a brain tumor found in a part of the brain stem called the pons. The pons controls essential bodily functions such as heartbeat, breathing, swallowing, eye movement, eyesight, and balance. DIPG affects children almost exclusively. Approximately 200-400 children in the United States are diagnosed with DIPG each year. These children are typically between the ages of 4 and 11. DIPG accounts for roughly 10-15% of all brain tumors in children. DIPG is an aggressive tumor that interferes with all bodily functions, depriving a child of the ability to move, to communicate, and even to eat and drink. Unfortunately, the prognosis for DIPGs is currently very poor.

Maternal embryonic leucine zipper kinase (MELK) is a serine/threonine kinase implicated in many cellular processes involved in embryogenesis and oncogenesis. Overexpression of MELK is a common feature of diffuse intrinsic pontine glioma (DIPG) and related to tumor grade. Inhibition of MELK by the small molecule OTS167 effectively inhibits migration, reduces proliferation and induces cell death in primary DIPG cell lines at low nanomolar concentrations. OTS167 co-inhibits neurotrophic tyrosine kinase, receptor-related 2 (ROR2), adding to a synergistic therapeutic effect in DIPG individuals that are characterized by overexpression of both targets.

Given the integrity of the blood-brain barrier in DIPG, an important consideration of any potential drug is its capacity to reach brain concentrations high enough for a therapeutic effect. Administration of the compound yields a brain-to-plasma (B/P) ratio estimated to be about 0.02. An absolute concentration of about 10 nanomolar can be reached in the brain with pharmaceutically acceptable toxic adverse effects, and is sufficient to induce apoptosis on glioma cells and leave healthy cells unharmed. This disclosure is directed to the compound represented by formula (1) with product name OTS167 and by a modified version of this compound represented by formula (2).

The present inventor has endeavored to develop a cure against Diffuse Intrinsic Pontine Glioma and has found that the use of a MELK inhibitor is effective in the treatment of DIPG, in particular, the use of a compound with product name OTS167 that inhibits target MELK (0.41 nM) and target ROR2 (50 - 100 nM), and that is represented by formula 1 or the SMILES description or a pharmaceutically acceptable compound thereof:

SMILES:CC(=O)c1cnc2ccc(nc2c1NC3CCC(CC3)CN(C)C)c4cc(c(c(c4)Cl)O)C 11-[6-(3,5-Dichloro-4-hydroxyphenyl)-4-({4-[(dimethylamino)methyl]cyclohexyl}amino)-1,5-naphthyridin-3-yl]ethanone

Molecular Formula C25H28Cl2N4O2

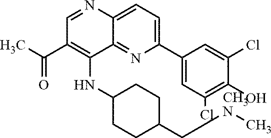 [SMILES: CC(=O)c1cnc2ccc(-c3cc(Cl)c(O)c(Cl)c3)nc2c1NC1CCC(CN(C)C)CC1]

Formula 1 (OTS167); 1-[6-(3,5-Dichloro-4-hydroxyphenyl)-4-({4-[(dimethylamino)methyl]cyclohexyl}amino)-1,5-naphthyridin-3-yl]ethanone

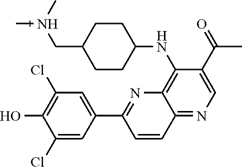 [SMILES: CC(=O)c1cnc2ccc(-c3cc(Cl)c(O)c(Cl)c3)nc2c1NC1CCC(C[NH+](C)C)CC1]

Formula 1 (OTS167); 1-[6-(3,5-Dichloro-4-hydroxyphenyl)-4-({4-[(dimethylamino)methyl]cyclohexyl}amino)-1,5-naphthyridin-3-yl]ethanone

### MELK

MELK, maternal embryonic leucine zipper kinase, was previously identified as a new member of the snfl /AMPK serine-threonine kinase family that is involved in mammalian embryonic development (Heyer BS et al, Dev Dyn. 1999 Aug 21 5(4):344-51). The gene was shown to play an important role in stem cell renewal (Nakano I et al., J Ceil Biol. 2005 Aug I, 170(3):413-27), cell-cycle progression (Blot J et al., Dev Biol. 2002 Jan 15, 241(2)i327-38; Seong HA et al, Biochem J. 2002 Feb 1, 361 (Pt 3): 597-604) and pre- mRNA splicing (Vdsieke V et a!., J Biol Chem. 2004 Mar 5, 279( i 0):8642-7. Epub 2003 Dec 29). in addition, through gene expression profile analysis using a genome-wide cDNA microarray containing 23,040 genes, MELK was recently shown to be up-regulated in breast cancer (Lin ML et al. Breast Cancer Res. 2007; 9 ( 1 ):R17, WO2006/016525, WO2008/023841). In fact, MELK is up-regulated in several cancer cells, for example, lung, bladder, lymphoma and cervical cancer cells (See WO2004/03 1413, WO20077013665, and WO2006/085684, the disclosures of which are incorporated by reference herein).

In most DIPG cell lines MELK is strongly overexpressed (WEE1 Kinase Inhibition Enhances the Radiation Response of Diffuse Intrinsic Pontine Gliomas Viola Caretti et. Al. J AACR 2012).

MELK and ROR2 targets (Ror2 as a Therapeutic Target in Cancer, Debebe at. al. Pharmacology and Therapeutics, 2015) are both primarily expressed during early embryogenesis. By Inhibiting these targets it is expected that no adverse effects or only very limited, pharmaceutical acceptable, adverse effects occur.

US9067937 B2 and US9345709 B2 describe 1,5-naphthyridine derivatives and MELK inhibitors containing the same that may be used in this disclosure.

Molecular dynamic computer simulations have shown that OTS167 inhibits MELK and co-inhibits to certain extend the Tyrosine-protein kinase transmembrane receptor ROR2 also known as neurotrophic tyrosine kinase, receptor-related 2, which is also overexpressed in primary DIPG cell lines (table 1). The MELK inhibitor OTS167 (IC₅₀ is 0.41 nM) might show a synergistic effect by co-inhibiting the ROR2 (IC₅₀ is calculated on 50 - 100 nM) target.

TABLE 1

Top 20 up-regulated kinases in DIPG

| Rank | Gene | Name | Probeset | Expression normal brain (log₂) | Expression DIPG (log₂) | Fold increase (log₂) | Pvalue (FDR) |
|---|---|---|---|---|---|---|---|
| 1 | TOP2A | Topoisomerase (DNA) II alpha | 201292_at | 1.05 | 7.85 | 7.45 | 3.30E-68 |
| 2 | MELK | Maternal embryonic leucine zipper kinase | 204825_at | 1.61 | 7.49 | 4.66 | 6.64E-62 |
| 3 | BUB1 | Budding uninhibited by benzimidazoles 1 homolog | 209642_at | 1.98 | 6.69 | 3.38 | 3.70E-44 |
| 4 | TTK | TTK protein kinase | 204822_at | 2.12 | 6.83 | 3.23 | 2.78E-51 |
| 5 | PBK | PDZ binding kinase | 219148_at | 2.46 | 7.61 | 3.10 | 5.90E-61 |
| 6 | OSR1 | Odd-skipped related 1 (Drosophila) | 228399_at | 1.87 | 5.66 | 3.03 | 7.03E-22 |
| 7 | WEE1 | WEE1 homolog | 212533_at | 3.60 | 8.81 | 2.45 | 2.60E-46 |
| 8 | NEK2 | NIMA (never in mitosis gene a)-related kinase 2 | 204641_at | 3.44 | 6.74 | 1.96 | 8.43E-24 |
| 9 | STK33 | Serine/threonine kinase 33 | 228035_at | 3.18 | 6.14 | 1.93 | 2.03E-19 |
| 10 | TEX14 | Testis expressed 14 | 221035_s_at | 2.92 | 5.50 | 1.88 | 5.24E-14 |
| 11 | CHEK1 | CHK1 checkpoint homolog (S. pombe) | 205394_at | 2.90 | 5.37 | 1.85 | 2.57E-30 |
| 12 | AURKB | Aurora kinase B | 209464_at | 3.05 | 5.32 | 1.74 | 2.45E-20 |
| 13 | BUB1B | Budding uninhibited by benzimidazoles 1 homolog beta (yeast) | 203755_at | 3.98 | 6.84 | 1.72 | 4.08E-32 |
| 14 | HK2 | Hexokinase 2 | 202934_at | 5.54 | 8.95 | 1.62 | 2.81E-32 |
| 15 | CHEK2 | CHK2 checkpoint homolog (S. pombe) | 210416_s_at | 3.51 | 5.43 | 1.55 | 3.41E-15 |
| 16 | AURKA | Aurora kinase A | 204092_s_at | 4.26 | 6.57 | 1.54 | 4.37E-33 |
| 17 | ROR2 | Receptor tyrosine kinase-like orphan receptor 2 | 205578_at | 2.41 | 3.71 | 1.54 | 3.62E-06 |
| 18 | PLAU | Plasminogen activator, urokinase | 205479_s_at | 3.52 | 5.41 | 1.54 | 2.29E-14 |
| 19 | DYRK3 | Dual-specificity tyrosine-(Y)-phosphorylation regulated kinase 3 | 210151_s_at | 4.28 | 6.32 | 1.48 | 6.32E-15 |
| 20 | MASTL | Microtubule-associated serine/threonine kinase-like | 228468_at | 4.21 | 6.13 | 1.46 | 3.76E-47 |

NOTE: Top 20 up-regulated kinases in DIPG tumor samples (n = 27; ref. 17) sorted on log2 fold increase as compared with nonmalignant brain tissues (n = 174; ref. 33) including 2 samples of normal brain stem tissue from the DIPG dataset (17). Wee1 homolog (WEE1) is identified as a highly differentially overexpressed kinase in DIPG.

Herein, with the term “overexpression” is meant at least 10, 20, 30, 40, 50% increased expression as compared to expression in normal brain, preferably as depicted above.

In most of DIPG tumor cell lines it has been shown that a concentration of 10 nM MELK inhibitor OTS167 is toxic and induces apoptosis on the primary tumor astrocytes, whereas the same doses of 10 nanomolar only slightly slows down the growth of normal astrocytes but no apoptosis occurs in non-somatic cells.

### OTS167 Clinical Dose and Maximum Tolerated Dose

OTSP167 is an extremely potent MELK inhibitor with IC₅₀ = 0.41 nM. To achieve a clinical effective dose of 15 micromolar solution in the blood plasma at a formula weight of 487.42 g/mol for OTS167, and an estimated 5 liters of blood plasma available in a child of age 5-12 years, the mass molarity calculation may pose a minimal dose 36.5 mg. The required minimal dose can be one order of magnitude smaller than the clinical dose or maximum tolerated dose for comparable kinase inhibiting compounds:

| | | | |
|---|---|---|---|
| Compound | MTD | Effect marker-based dose | Clinical dose |
| Cetuximab | Not reached | 200 mg/^{m2} q1w | 250 mg/m² q1w as maintenance dose (initial dose = 400 mg/m2) |
| Pembrolizumab | Not reached up to 10 mg/kg q2w | 2 mg/kg q3w | 2 mg/kg q3w |
| Idelalisib | Not reached up to 350 mg twice a day | 150 mg twice a day | 150 mg twice a day |
| Decitabine | 1,500-2,000/mg/m² over 1-3 days | 15-20 mg/m²/d over 10 days | 20 mg/m²/d over 5 days |
| Vismodegib | Not reached up to 540 mg/d | 150 mg/d | 150 mg/d |
| Crizotinib | 250 mg twice a day | 250 mg twice a day; pharmacodynamic marker helped in target population selection | 250 mg twice a day (Alk-positive patients) |
| Galunisertib | Not able to determine MTD due to potential cardiovascular DLT | 160-360 mg/d with intermittent dosing (2 weeks on and 2 weeks off) | Not approved yet; pharmacokinetic-pharmacodynamic model-based intermittent dosing enabled clinical development |

Abbreviations: q1w, every week; q2w, every 2 weeks; q3w, every 3 weeks. Table 2: Clin. Cancer Res; 22(6) Mar. 15, 2016

Another example of the excellent MTB outcome for OTS167 is the comparison with the clinical evaluation of the AZD1152 inhibitor on the serine/threonine kinase Aurora B, in which the MTB is, dependent on the dosing schedule, 200-450 mg.

### BRIEF DESCRIPTION OF THE DRAWING

FIG. 1 is a graph that shows the calculated Blood-brain barrier crossing of the modified OTS167 (ID: FOLDYNE-1332-A) compound.

### DETAILED DESCRIPTION

The objective of the disclosure is to contribute to a better prognosis in DIPG.

### OTS167 and its Passive or Active Transport Over the Blood-Brain Barrier

Passive transport of OTS167 over the blood-brain barrier is possible in mouse models (wild type), brain-to-plasma (B/P) ratio = 0.02. Experiments have shown that the inhibitor can be transported actively over the blood-brain barrier preferably by co-administering a P-glycoprotein1 inhibitor, or ATP-binding cassette sub-family B member 1 (Abcb1a) inhibitor, increasing the concentration of OTS167 in the brain tissue by at least a factor of 4 and showing pharmaceutical acceptable adverse effects.

Inhibition of other ABC transporters might increase the concentration of OTS 167 in the brain tissue. The ABC transporters are Abcb1a, Abcb1b and Abcg2.

In addition, mannitol can be used in bypassing the blood-brain barrier.

### Administering OTS167 for Diffuse Intrinsic Pontine Glioma

The MELK inhibitor according to this disclosure (i.e., the (modified) OTS167), can be applied in the form of solutions, e.g., aqueous solutions, advantageously intravenously, e.g., as a suspension or in aqueous solution. The dosage in vitro may range between about 10-3 molar and 10-9 molar concentrations. A therapeutically effective amount in vivo may range depending on the route of administration, between about 1-500 mg/kg, typically 10-100 mg/kg. When administered orally, a dose of 100-300 or about 200 mg/kg is suitable for the treatment of Diffuse Intrinsic Pontine Glioma.

### OTS167 and Convection Enhanced Delivery

OTS167 can be delivered through one to several catheters placed stereotactically directly within the pontine glioma tumor tissue. OTS167 shows a well spread distribution through convection or molecular diffusion within the tumor tissue.

### High-Intensity Focused Ultrasound

Stimulation in blood-brain barrier crossing of described compounds can be obtained by mediation of high-intensity focused ultrasound in the ranges 500 KHz to 1.5 MHz causing sonoporation and/or sonopermeabilization in the tight junction.

OTS167 might be administered (as stand-alone compound) below toxic levels, comparable with typical concentrations of vitamins (approximately 20 micromoles/liter for vitamin E, or 50 micromoles/liter for vitamin C) in the blood plasma. The compound might yield at lethal levels of at least 10 nM concentration in the brain tumor tissue, by a blood plasma-driven chemical potential of 0.5 µM only.

### Modified OTS167 Crosses the Blood-Brain Barrier and Binds Even Stronger to MELK Than OTS167

A chemical modification of the OTS167 molecule of a specific atom N to C decreases the IC₅₀ on the MELK target to less than 0.41 nanomolar. The stronger binding occurs due to the N to C substitution and is caused by efficient expulsion of a high energetic, residing water molecule in the active site. The energy of binding from the compound to the target is increased. However, it is of paramount importance that due to increased lipophilicity at the most optimal position within the chemical structure, the compound is predicted to pass fluently through the brain barrier.

Semi-empirical quantum chemical calculations show that the OTS167 molecule possesses an excess positive charge in solution, hindering its passage through the blood brain barrier, while the most dominant tautomer of the modified version of OTS 167 is neutral in charge, yielding strongly increased lipophilicity. While the unsubstituted N atom of the OTS167 molecule does not form an energetically favorable hydrogen bridge with the Melk target, the modified OTS167 molecule yields increased desolvation energy, contributing to stronger binding energy, however the atomic substitution does not contribute to better solubility in water.

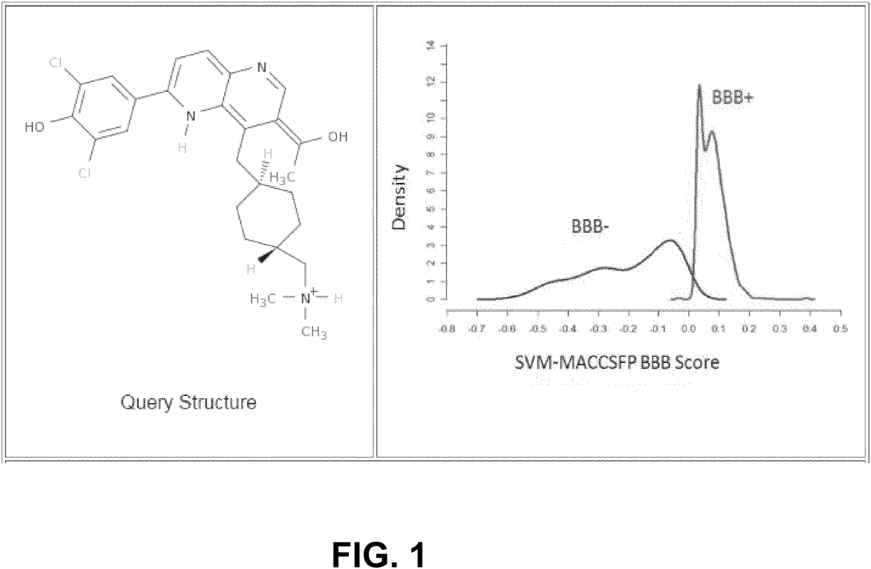

FIG. 1 shows the calculated Blood-brain barrier crossing of the modified OTS167 (ID: FOLDYNE-1332-A) compound: brain-to-plasma (B/P) SVM_MACCSFP BBB score = 0.025. That score is only a factor 4 smaller compared with the score of 0.1 for melatonin or ethanol, which permeate perfectly through the BBB. Administering of the OTS167 compound yields a brain-to-plasma (B/P) ratio of 0.02 in mouse models. Calculated value of BBB-/BBB+ ratio as described herein concerns crossing over the tight junction in the barrier between the endothelial cells. Left image: the SVM_MACCSFP BBB score is 0.25. Right image: Threshold of BBB-/BBB+ Score is 0.02. The compound is predicted as BBB+.

The N to C atomic substitution of the modified OTS167, which is located in the center of the molecule, and the center of the targeted active site, has profound implications on bond lengths and atom angles of adjacent atoms in OTS167. In addition, the dihedral angles of all 4 atom combinations in which the N to C substitution participates, have a different dihedral potential energy profile resulting in a lower intramolecular energy. All described physical properties favor the energy of binding of the N to C substitution in the modified OTS 167 version to the MELK target.

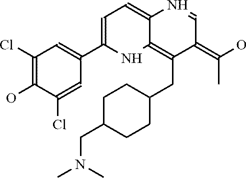 [SMILES: C/C(O)=c1/cnc2c(c1CC1CCC(CN(C)C)CC1)NC(c1cc(Cl)c(O)c(Cl)c1)=CC=2]

Modified OTS167 is any of the above or preferably:

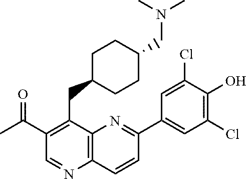 [SMILES: CC(=O)c1cnc2ccc(-c3cc(Cl)c(O)c(Cl)c3)nc2c1C[C@H]1CC[C@H](CN(C)C)CC1]

Modified OTS167 (formula 2) or
CC(O)=c3cnc2ccc(c1cc(Cl)c(O)c(Cl)c1)[nH]c2c3CC4CCC(CN(C)C)CC4

(ID: FOLDYNE-1332-A)

Below, the N to C substitution with respect to unmodified OTS167 has been specified:

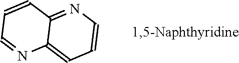

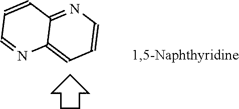

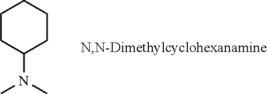

The Nitrogen atom connecting the 1,5-Naphthyridine group (first structure) and N,N-Dimethylcyclohexanamine group (second structure) into:

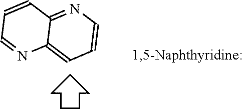

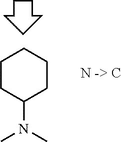

N,N-Dimethylcyclohexanamine Into:

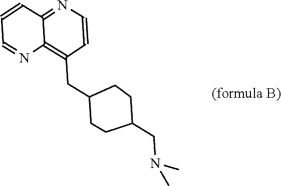 [SMILES: CN(C)CC1CCC(Cc2ccnc3cccnc23)CC1]

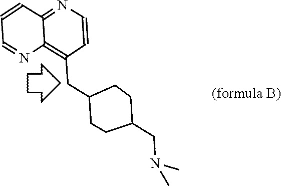 [SMILES: CN(C)CC1CCC(Cc2ccnc3cccnc23)CC1]

### Multiple Kinase Binding in DIPG

Additionally contributing to DIPG inhibiting selectivity, the modified OTS167 version has, according to molecular dynamics simulation, a strongly increased energy of binding to the following targets, present in Table 1: Top 20 up-regulated kinases in DIPG and ranked at positions 3 and 12; Mitotic checkpoint serine/threonine protein kinase (gene BUB1) and Aurora kinase type B (gene AURKB).

The modes of binding of the modified OTS167 molecule to the targets BUB1 and AURKB are similar to the binding mode in the MELK target, and involve binding to contact residues in the kinase active site binding pockets with strongly homologous residue sequences relative to the MELK kinase.

### Increased Lipophilicity of Modified OTS167 and Strategy of Drug Delivery

The high lipophilicity and relatively low molecular weight of modified OTS167 enables the pathway strategy of intranasal administration. Generally, lipophilic drugs are strongly absorbed from the nasal cavity compared to polar drugs and the bioavailability could approach 100%, and in addition, the nasal route avoids hepatic first pass elimination associated with oral delivery. The direct connection between the brain stem and nasal mucosa through cranial nerve pathways allows direct delivery of modified OTS167. When modified OTS167 is administered in the nasal olfactory region the blood-brain barrier is optimally circumvented.

The OTS167 molecule is calculated to yield comparable bioavailability with modified OTS167 through the described nasal pathway if formulated with multiple units of β-(1→4)-linked D-glucosamine and N-acetyl-D-glucosamine (Chitosan). The formulation might also further enhance the delivery of modified OTS167.

### the Synthesis of Modified OTS167

The synthesis of modified OTS167 is depicted in the schemes below.

The procedure for the synthesis of OTS167 is described in the patent (WO2013109388A2). In modified OTS167, a methylenecyclohexane group can be used as main partial intermediate. Two routes are being proposed based on similar chemistry known in the literature. The first route (Scheme 1+2) is based on the chemistry reported in Bioorg. Med. Chem. Lett. 1998, 8, 2813 where a phenol group is being transformed into alkyl group via triflate intermediate in the presence of Zn dust and Pd(dba) catalyst.

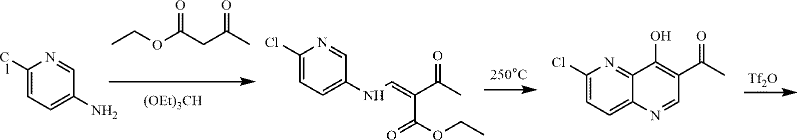

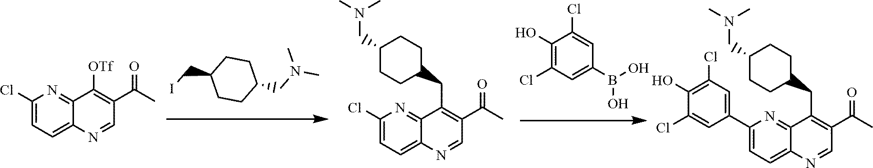

Scheme 1

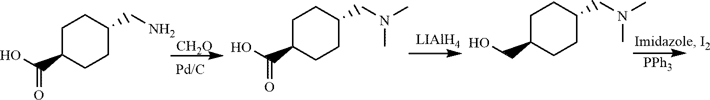

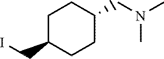

### Scheme 2

The second route (Scheme 3) is based on the Kumada coupling between aryl halide and alkylmagnesium chloride compound giving key intermediate 1a.

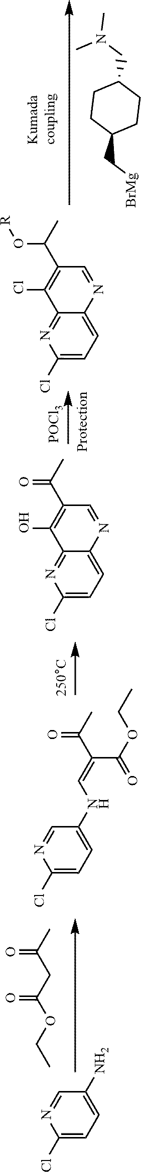

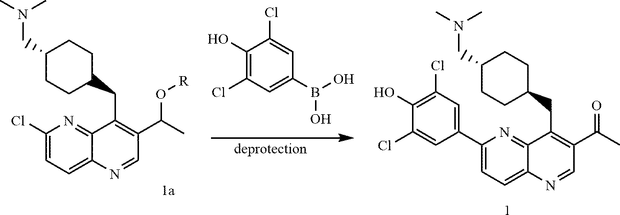

### Scheme 3

In this disclosure and/or claims, as the MELK inhibitor (or as the (modified) OTS167 molecule) may be used a molecule as disclosed in US9067937 or US9345709 or according to the following clauses:

1. A compound represented by formula (I) or a pharmaceutically acceptable salt thereof:

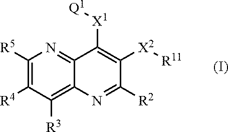

wherein,
X¹ is selected from the group consisting of a direct bond, —NR¹²—, —O—, and —S—;
R¹² is selected from the group consisting of a hydrogen atom, C₁-C₆ alkyl and C₃-C₁₀ cycloalkyl;
Q¹ is selected from the group consisting of C₃-C₁₀ cycloalkyl, C₆-C₁₀ aryl, 5- to 10-membered heteroaryl, 3- to 10-membered non-aromatic heterocyclyl, (C₃-C₁₀ cycloalkyl)-C₁-C₆ alkyl, (C₆-C₁₀ aryl)-C₁-C₆ alkyl, (5- to 10-membered heteroaryl)-C₁-C₆ alkyl, and (3-to 10-membered non-aromatic heterocyclyl)-C₁-C₆ alkyl; wherein Q¹ is optionally substituted with one or more substituents independently selected from A¹;
X² is selected from the group consisting of —CO—, —S—, —SO—, and —SO₂—;
R¹¹ is selected from the group consisting of C₁-C₆ alkyl, C₃-C₁₀ cycloalkyl, C₆-C₁₀ aryl, 5- to 10-membered heteroaryl, and 3- to 10-membered non-aromatic heterocyclyl; wherein R¹¹ is optionally substituted with one or more substituents independently selected from A²;
R⁵ is selected from the group consisting of a halogen atom, C₃-C₁₀ cycloalkyl, C₆-C₁₀ aryl, 5- to 10-membered heteroaryl, and 3- to 10-membered non-aromatic heterocyclyl; wherein the cycloalkyl, aryl, heteroaryl, and heterocyclyl are optionally substituted with one or more substituents independently selected from A³;
R², R³, and R⁴ are independently selected from the group consisting of a hydrogen atom, a halogen atom, and C₁-C₆ alkyl;
A¹ and A³ are independently selected from the group consisting of a halogen atom, cyano, —COOR¹³, —CONR¹⁴R¹⁵, formyl, (C₁-C₆ alkyl)carbonyl, C₁-C₆ alkyl, C₂-C₆ alkenyl, C₂-C₆ alkynyl, nitro, —NR¹⁶R¹⁷, —OR¹⁸, —S(O)ₙR¹⁹, C₃-C₁₀ cycloalkyl, C₆-C₁₀ aryl, 5- to 10-membered heteroaryl, and 3- to 10-membered non-aromatic heterocyclyl; wherein the alkylcarbonyl, alkyl, alkenyl, alkynyl, cycloalkyl, aryl, heteroaryl, and heterocyclyl are optionally substituted with one or more substituents independently selected from A⁴;
A² is independently selected from the group consisting of a halogen atom, cyano, C₃-C₁₀ cycloalkyl, carboxy, formyloxy, (C₁-C₆ alkyl)carbonyloxy, hydroxy, C₁-C₆ alkoxy, amino, C₁-C₆ alkylamino, and di(C₁-C₆ alkyl)amino;
R¹³, R¹⁴, and R¹⁵ are independently selected from the group consisting of a hydrogen atom, C₁-C₆ alkyl, C₂-C₆ alkenyl, C₂-C₆ alkynyl, C₃-C₁₀ cycloalkyl, C₆-C₁₀ aryl, 5-to 10-membered heteroaryl, and 3- to 10-membered non-aromatic heterocyclyl; wherein the alkyl, alkenyl, alkynyl, cycloalkyl, aryl, heteroaryl, and heterocyclyl are optionally substituted with one or more substituents independently selected from A⁴; or R¹⁴ and R¹⁵ together with the nitrogen atom to which they are attached form 3- to 10-membered nitrogen-containing heterocyclyl, which is optionally substituted with one or more substituents independently selected from A⁴;
R¹⁶ and R¹⁸ are independently selected from the group consisting of a hydrogen atom, C₁-C₆ alkyl, C₂-C₆ alkenyl, C₂-C₆ alkynyl, C₃-C₁₀ cycloalkyl, C₆-C₁₀ aryl, 5- to 10-membered heteroaryl, 3- to 10-membered non-aromatic heterocyclyl, and —COR²⁰; wherein the alkyl, alkenyl, alkynyl, cycloalkyl, aryl, heteroaryl, and heterocyclyl are optionally substituted with one or more substituents independently selected from A⁴; R¹⁷ is selected from the group consisting of a hydrogen atom, and C₁-C₆ alkyl that is optionally substituted with one or more substituents independently selected from A⁴; or R¹⁶ and R¹⁷ together with the nitrogen atom to which they are attached form 3- to 10-membered nitrogen-containing heterocyclyl, which is optionally substituted with one or more substituents independently selected from A⁴;
R¹⁹ is selected from the group consisting of C₁-C₆ alkyl, C₃-C₁₀ cycloalkyl, C₆-C₁₀ aryl, and 5- to 10-membered heteroaryl; wherein the alkyl, cycloalkyl, aryl, and heteroaryl are optionally substituted with one or more substituents independently selected from A⁴;
R²⁰ is selected from the group consisting of a hydrogen atom, -NR¹⁴R¹⁵, C₁-C₆ alkyl, C₂-C₆ alkenyl, C₂-C₆ alkynyl, C₃-C₁₀ cycloalkyl, C₆-C₁₀ aryl, 5- to 10-membered heteroaryl, and 3- to 10-membered non-aromatic heterocyclyl; wherein the alkyl, alkenyl, alkynyl, cycloalkyl, aryl, heteroaryl, and heterocyclyl are optionally substituted with one or more substituents independently selected from A⁴;
n is an integer independently selected from 0 to 2;
A⁴ is independently selected from the group consisting of a halogen atom, cyano, — COOR²¹, —CONR²²R²³, formyl, (C₁-C₆ alkyl)carbonyl, C₁-C₆ alkyl, C₂-C₆ alkenyl, C₂-C₆ alkynyl, nitro, -NR²⁴R²⁵, —OR²⁶, —S(O)ₙR²⁷, C₃-C₁₀ cycloalkyl, C₆-C₁₀ aryl, 5- to 10-membered heteroaryl, and 3- to 10-membered non-aromatic heterocyclyl; wherein the alkylcarbonyl, alkyl, alkenyl, alkynyl, cycloalkyl, aryl, heteroaryl, and heterocyclyl are optionally substituted with one or more substituents independently selected from A⁵;
R²¹, R²², and R²³ are independently selected from the group consisting of a hydrogen atom, C₁-C₆ alkyl, C₂-C₆ alkenyl, C₂-C₆ alkynyl, C₃-C₁₀ cycloalkyl, C₆-C₁₀ aryl, 5-to 10-membered heteroaryl, and 3- to 10-membered non-aromatic heterocyclyl; wherein the alkyl, alkenyl, alkynyl, cycloalkyl, aryl, heteroaryl, and heterocyclyl are optionally substituted with one or more substituents independently selected from A⁵; or R²² and R²³ together with the nitrogen atom to which they are attached form 3- to 10-membered nitrogen-containing heterocyclyl, which is optionally substituted with one or more substituents independently selected from A⁵;
R²⁴ and R²⁶ are independently selected from the group consisting of a hydrogen atom, C₁-C₆ alkyl, C₂-C₆ alkenyl, C₂-C₆ alkynyl, C₃-C₁₀ cycloalkyl, C₆-C₁₀ aryl, 5- to 10-membered heteroaryl, 3- to 10-membered non-aromatic heterocyclyl, and -COR²⁸; wherein the alkyl, alkenyl, alkynyl, cycloalkyl, aryl, heteroaryl, and heterocyclyl are optionally substituted with one or more substituents independently selected from A⁵; R²⁵ is selected from the group consisting of a hydrogen atom, and C₁-C₆ alkyl that is optionally substituted with one or more substituents independently selected from A⁵; or R²⁴ and R²⁵ together with the nitrogen atom to which they are attached form 3- to 10-membered nitrogen-containing heterocyclyl, which is optionally substituted with one or more substituents independently selected from A⁵;
R²⁷ is selected from the group consisting of C₁-C₆ alkyl, C₃-C₁₀ cycloalkyl, C₆-C₁₀ aryl, and 5- to 10-membered heteroaryl; wherein the alkyl, cycloalkyl, aryl, and heteroaryl are optionally substituted with one or more substituents independently selected from A⁵;
R²⁸ is independently selected from the group consisting of a hydrogen atom, —NR²²R²³, C₁-C₆ alkyl, C₂-C₆ alkenyl, C₂-C₆ alkynyl, C₃-C₁₀ cycloalkyl, C₆-C₁₀ aryl, 5- to 10-membered heteroaryl, and 3- to 10-membered non-aromatic heterocyclyl; wherein the alkyl, alkenyl, alkynyl, cycloalkyl, aryl, heteroaryl, and heterocyclyl are optionally substituted with one or more substituents independently selected from A⁵;
A⁵ is independently selected from the group consisting of a halogen atom, cyano, — COOR³¹, —CONR³²R³³, formyl, (C₁-C₆ alkyl)carbonyl, C₁-C₆ alkyl, C₂-C₆ alkenyl, C₂-C₆ alkynyl, nitro, -NR³⁴R³⁵, —OR³⁶, —S(O)ₙR³⁷, C₃-C₁₀ cycloalkyl, C₆-C₁₀ aryl, 5- to 10-membered heteroaryl, and 3- to 10-membered non-aromatic heterocyclyl; wherein the alkylcarbonyl, alkyl, alkenyl, alkynyl, cycloalkyl, aryl, heteroaryl, and heterocyclyl are optionally substituted with one or more substituents independently selected from A⁶;
R³¹, R³², and R³³ are independently selected from the group consisting of a hydrogen atom, C₁-C₆ alkyl, C₂-C₆ alkenyl, C₂-C₆ alkynyl, C₃-C₁₀ cycloalkyl, C₆-C₁₀ aryl, 5-to 10-membered heteroaryl, and 3- to 10-membered non-aromatic heterocyclyl; wherein the alkyl, alkenyl, alkynyl, cycloalkyl, aryl, heteroaryl, and heterocyclyl are optionally substituted with one or more substituents independently selected from A⁶; or R³² and R³³ together with the nitrogen atom to which they are attached form 3- to 10-membered nitrogen-containing heterocyclyl, which is optionally substituted with one or more substituents independently selected from A⁶;
R³⁴ and R³⁶ are independently selected from the group consisting of a hydrogen atom, C₁-C₆ alkyl, C₂-C₆ alkenyl, C₂-C₆ alkynyl, C₃-C₁₀ cycloalkyl, C₆-C₁₀ aryl, 5- to 10-membered heteroaryl, 3- to 10-membered non-aromatic heterocyclyl, and -COR³⁸; wherein the alkyl, alkenyl, alkynyl, cycloalkyl, aryl, heteroaryl, and heterocyclyl are optionally substituted with one or more substituents independently selected from A⁶; R³⁵ is selected from the group consisting of a hydrogen atom, and C₁-C₆ alkyl that is optionally substituted with one or more substituents independently selected from A⁶; or R³⁴ and R³⁵ together with the nitrogen atom to which they are attached form 3- to 10-membered nitrogen-containing heterocyclyl, which is optionally substituted with one or more substituents independently selected from A⁶;
R³⁷ is selected from the group consisting of C₁-C₆ alkyl, C₃-C₁₀ cycloalkyl, C₆-C₁₀ aryl, and 5- to 10-membered heteroaryl; wherein the alkyl, cycloalkyl, aryl, and heteroaryl are optionally substituted with one or more substituents independently selected from A⁶;
R³⁸ is independently selected from the group consisting of a hydrogen atom, - NR³²R³³, C₁-C₆ alkyl, C₂-C₆ alkenyl, C₂-C₆ alkynyl, C₃-C₁₀ cycloalkyl, C₆-C₁₀ aryl, 5- to 10-membered heteroaryl, and 3- to 10-membered non-aromatic heterocyclyl; wherein the alkyl, alkenyl, alkynyl, cycloalkyl, aryl, heteroaryl, and heterocyclyl are optionally substituted with one or more substituents independently selected from A⁶;
A⁶ is independently selected from the group consisting of a halogen atom, cyano, carboxy, —COOR⁴¹, —CONR⁴²R⁴³, formyl, (C₁-C₆ alkyl)carbonyl, C₁-C₆ alkyl, C₂-C₆ alkenyl, C₂-C₆ alkynyl, nitro, —NR⁴⁴R⁴⁵, —OR⁴⁶, S(O)ₙR⁴⁷, C₃-C₁₀ cycloalkyl, C₆-C₁₀ aryl, 5- to 10-membered heteroaryl, and 3- to 10-membered non-aromatic heterocyclyl; wherein the alkylcarbonyl, alkyl, alkenyl, alkynyl, cycloalkyl, aryl, heteroaryl, and heterocyclyl are optionally substituted with one or more substituents independently selected from the group consisting of a halogen atom, hydroxy, C₁-C₆ alkoxy, amino, C₁-C₆ alkylamino, and di(C₁-C₆ alkyl)amino;
R⁴¹, R⁴², and R⁴³ are independently selected from the group consisting of a hydrogen atom, C₁-C₆ alkyl, C₂-C₆ alkenyl, C₂-C₆ alkynyl, C₃-C₁₀ cycloalkyl, C₆-C₁₀ aryl, 5-to 10-membered heteroaryl, and 3- to 10-membered non-aromatic heterocyclyl; wherein the alkyl, alkenyl, alkynyl, cycloalkyl, aryl, heteroaryl, and heterocyclyl are optionally substituted with one or more substituents independently selected from the group consisting of a halogen atom, hydroxy, C₁-C₆ alkoxy, amino, C₁-C₆ alkylamino, and di(C₁-C₆ alkyl)amino;
R⁴⁴ and R⁴⁶ are independently selected from the group consisting of a hydrogen atom, C₁-C₆ alkyl, C₃-C₁₀ cycloalkyl, C₆-C₁₀ aryl, 5- to 10-membered heteroaryl, 3- to 10-membered non-aromatic heterocyclyl, and —COR⁴⁸;
R⁴⁵ is selected from the group consisting of a hydrogen atom, and C₁-C₆ alkyl;
R⁴⁷ is selected from the group consisting of C₁-C₆ alkyl, C₃-C₁₀ cycloalkyl, C₆-C₁₀ aryl, and 5- to 10-membered heteroaryl; and
R⁴⁸ is independently selected from the group consisting of C₁-C₆ alkyl, C₃-C₁₀ cycloalkyl, C₆-C₁₀ aryl, 5- to 10-membered heteroaryl, and 3- to 10-membered non-aromatic heterocyclyl.

2. The compound or a pharmaceutically acceptable salt thereof according to clause 1, wherein Q¹ is selected from the group consisting of C₅-C₇ cycloalkyl, phenyl, pyridyl, pyrazolyl, pyrimidinyl, and piperidyl; wherein Q¹ is optionally substituted with one or more substituents independently selected from A¹.

3. The compound or a pharmaceutically acceptable salt thereof according to clause 1 or 2, wherein X² is selected from the group consisting of —CO— and —SO₂—; and R¹¹ is selected from the group consisting of C₁-C₆ alkyl and C₃-C₇ cycloalkyl, which are optionally substituted with one or more substituents independently selected from the group consisting of hydroxy and a halogen atom.

4. The compound or a pharmaceutically acceptable salt thereof according to any one of clauses 1 to 3, wherein R⁵ is phenyl substituted with one to three substituents independently selected from the group consisting of hydroxy, a halogen atom, C₁-C₆ alkyl, and C₁-C₆ alkoxy, wherein the alkyl and alkoxy are optionally substituted with one or more halogen atoms.

5. The compound or a pharmaceutically acceptable salt thereof according to any one of clauses 1 to 4, wherein R² is a hydrogen atom.

6. The compound or a pharmaceutically acceptable salt thereof according to any one of clauses 1 to 5, wherein R³ is a hydrogen atom.

7. The compound or a pharmaceutically acceptable salt thereof according to any one of clauses 1 to 6, wherein R⁴ is a hydrogen atom.

8. The compound or a pharmaceutically acceptable salt thereof according to any one of clauses 1 to 7, wherein X¹ is —NH—.

9. The compound or a pharmaceutically acceptable salt thereof according to any one of clauses 1 to 8, wherein the optional substituent of Q¹ is selected from the group consisting of hydroxy, amino, C₁-C₆ alkoxy, C₁-C₆ alkylamino, di(C₁-C₆ alkyl)amino, amino-C₁-C₆ alkyl, (C₁-C₆ alkylamino)-C₁-C₆ alkyl, di(C₁-C₆ alkyl)amino-C₁-C₆ alkyl, amino-C₁-C₆ alkoxy, (C₁-C₆ alkylamino)-C₁-C₆ alkoxy, di(C₁-C₆ alkyl)amino-C₁-C₆ alkoxy, hydroxy-C₁-C₆ alkyl, (C₁-C₆ alkoxy)-C₁-C₆ alkyl, carboxy-C₁-C₆ alkyl, [(C₁-C₆ alkoxy)carbonyl]-C₁-C₆ alkyl, carbamoyl-C₁-C₆ alkyl, [N-(C₁-C₆ alkyl) carbamoyl]-C₁-C₆ alkyl, [N,N-di(C₁-C₆ alkyl)carbamoyl]-C₁-C₆ alkyl, (C₁-C₆ alkyl)carbonylamino, N-(C₁-C₆ alkyl)carbonyl-N-(C₁-C₆ alkyl)amino, pyrrolidinyl, piperidyl, piperazinyl; wherein the pyrrolidinyl, piperidyl, and piperazinyl defined as the optional substituent of Q¹ are optionally substituted with a substituent selected from the group consisting of C₁-C₆ alkyl, amino, C₁-C₆ alkylamino, di(C₁-C₆ alkyl)amino, hydroxy, C₁-C₆ alkoxy, pyrrolidinyl, piperidyl, and piperazinyl; and wherein the alkyl moiety of the group defined as the optional substituent of Q¹ is optionally substituted with a substituent selected from the group consisting of amino, C₁-C₆ alkylamino, di(C₁-C₆ alkyl)amino, hydroxy, C₁-C₆ alkoxy, pyrrolidinyl, piperidyl, and piperazinyl.

10. The compound or a pharmaceutically acceptable salt thereof according to clause 9, wherein the optional substituent of Q¹ is selected from the group consisting of hydroxy, amino, di(C₁-C₆ alkyl)amino, C₁-C₆ alkyl, di(C₁-C₆ alkyl)amino-C₁-C₆ alkyl, di(C₁-C₆ alkyl)amino-C₁-C₆ alkoxy, di(C₁-C₆ alkyl)amino, [(amino-C₁-C₆ alkyl)carbonyl]amino, N-(C₁-C₆ alkyl)piperidyl, di(C₁-C₆ alkyl)amino-pyrrolidin-1-yl, amino-pyrrolidin-1-yl, (pyrrolidin-1-yl)-C₁-C₆ alkyl, (C₁-C₆ alkyl)amino-piperidin-1-yl, amino-piperidin-1-yl, hydroxy-C₁-C₆ alkyl, [di(C₁-C₆ alkyl)amino-C₁-C₆ alkyl]amino, [4-(C₁-C₆ alkyl)-piperazin-1-yl]-C₁-C₆ alkyl, (piperazin-1-yl)-C₁-C₆ alkyl, pyrrolidinylcarbonyl-amino, (hydroxy-pyrrolidin-1-yl)-C₁-C₆ alkyl, morpholino-C₁-C₆ alkyl, [N-(hydroxy-C₁-C₆ alkyl)-N-(C₁-C₆ alkyl)amino]-C₁-C₆ alkyl, and (CD₃)₂N-C₁-C₆ alkyl.

11. The compound or a pharmaceutically acceptable salt thereof according to clause 1, which is selected from the group consisting of the following compounds:
1-(6-chloro-4-(4-((dimethylamino)methyl) cyclohexylamino)-1,5-naphthyridin- 3-yl)ethanone;
1-(6-(3,5-dichloro-4-hydroxyphenyl)-4-((4-(dimethylamino)cyclohexyl)amino)-1,5-naphthyridin-3-yl)ethanone;
1-(6-(3-chloro-5-fluoro-4-hydroxyphenyl)-4-((4-(dimethylamino)cyclohexyl)amino)-1,5-naphthyridin-3-yl)ethanone;
cyclopropyl(6-(3,5-dichloro-4-hydroxyphenyl)-4-(4-((dimethylamino)methyl)-cyclohexylamino)-1,5-naphthyridin-3-yl)methanone;
(6-(3-chloro-5-fluoro-4-hydroxyphenyl)-4-(4-((dimethylamino)methyl)-cyclohexylamino)-1,5-naphthyridin-3-yl)(cyclopropyl)methanone;
1-(6-(3,5-dichloro-4-hydroxyphenyl)-4-((4-((dimethylamino)methyl)cyclohexyl)-amino)-1,5-naphthyridin-3-yl)ethanone;
1-(6-(3 -chloro-5 -fluoro-4-hydroxyphenyl)-4-((4-((dimethylamino)methyl)cyclohexyl)-amino)-1,5-naphthyridin-3-yl)ethanone;
1-(6-(3 -chloro-4-hydroxy-5 -methoxyphenyl)-4-((4-((dimethylamino)methyl)-cyclohexyl)amino)-1,5-naphthyridin-3-yl)ethanone;
1-(6-(3,5-dichloro-4-hydroxyphenyl)-4-((4-(2-(dimethylamino)ethyl)cyclohexyl)-amino)-1,5-naphthyridin-3-yl)ethanone;
1-(6-(3 -chloro-5 -fluoro-4-hydroxyphenyl)-4-(4-(2-(dimethylamino)ethyl)-cyclohexylamino)-1,5-naphthyridin-3-yl)ethanone;
1-(4-(4-((dimethylamino)methyl)cyclohexylamino)-6-(4-hydroxy-3-(trifluoromethoxy)- phenyl)-1,5-naphthyridin-3-yl)ethanone;
2,6-dichloro-4-(8-((4-((dimethylamino)methyl)cyclohexyl)amino)-7-(methyl sulfonyl)-1,5 -naphthyridin-2-yl)phenol;
2-chloro-4-(8-((4-((dimethylamino)methyl)cyclohexyl)amino)-7-(methyl sulfonyl)-1,5 -naphthyridin-2-yl)-6-fluorophenol;
2-chloro-4-(8-((4-((dimethylamino)methyl)cyclohexyl)amino)-7-(methyl sulfonyl)-1,5-naphthyridin-2-yl)-6-methoxyphenol;
2,6-dichloro-4-(8-((4-(dimethylamino)cyclohexyl)amino)-7-(methyl sulfonyl)-1,5 -naphthyridin-2-yl)phenol;
2,6-dichloro-4-(8-((4-((dimethylamino)methyl)phenyl)amino)-7-(methylsulfonyl)-1,5- naphthyridin-2-yl)phenol;
2-chloro-4-(8-((4-((dimethylamino)methyl)phenyl)amino)-7-(methylsulfonyl)-1,5-naphthyridin-2-yl)-6-fluorophenol;
2-chloro-4-(8-((4-((dimethylamino)methyl)phenyl)amino)-7-(methyl sulfonyl)-1,5-naphthyridin-2-yl)-6-methoxyphenol;
1-(6-(3,5-dichloro-4-hydroxyphenyl)-4-((3-(2-(pyrrolidin-1-yl)ethyl)phenyl)amino)-1,5- naphthyridin-3-yl)ethanone;
1-(6-(3-chloro-5-fluoro-4-hydroxyphenyl)-4-(3-(2-(pyrrolidin-1-yl)ethyl)phenylamino)-1,5-naphthyridin-3 -yl)ethanone;
1-(6-(3,5-dichloro-4-hydroxyphenyl)-4-((6-(2-(dimethylamino)ethoxy)pyridin-3-yl)- amino)-1,5-naphthyridin-3-yl)ethanone;
1-(6-(3-chloro-5-fluoro-4-hydroxyphenyl)-4-((6-(2-(dimethylamino)ethoxy)pyridin-3- yl)amino)-1,5-naphthyridin-3-yl)ethanone;
1-(6-(3-chloro-4-hydroxy-5-methoxyphenyl)-4-((6-(2-(dimethylamino)ethoxy)pyridin-3-yl)amino)-1,5-naphthyridin-3-yl)ethanone;
2,6-dichloro-4-(8-((6-(2-(dimethylamino)ethoxy)pyridin-3-yl)amino)-7-(methyl sulfonyl)-1,5 -naphthyridin-2-yl)phenol;
2-chloro-4-(8-((6-(2-(dimethylamino)ethoxy)pyridin-3-yl)amino)-7-(methylsulfonyl)- 1,5-naphthyridin-2-yl)-6-fluorophenol;
2-chloro-4-(8-((6-(2-(dimethylamino)ethoxy)pyridin-3-yl)amino)-7-(methylsulfonyl)- 1,5-naphthyridin-2-yl)-6-methoxyphenol;
1-(6-(3,5-dichloro-4-hydroxyphenyl)-4-((1-methylpiperidin-4-yl)methylamino)-1,5-naphthyridin-3-yl)ethanone;
1-(6-(3,5-dichloro-4-hydroxyphenyl)-4-((4-((dimethylamino-d6)methyl)cyclohexyl)- amino)-1,5-naphthyridin-3 -yl)ethanone;
1-(6-(3,5-dichloro-4-hydroxyphenyl)-4-((4-(2-(dimethylamino)ethyl)phenyl)amino)- 1,5-naphthyridin-3-yl)ethanone;
1-(6-(3-chloro-5-fluoro-4-hydroxyphenyl)-4-((4-(2-(dimethylamino)ethyl)phenyl)-amino)-1,5-naphthyridin-3-yl)ethanone;
1-(6-(3-chloro-4-hydroxy-5-methoxyphenyl)-4-((4-(2-(dimethylamino)ethyl)phenyl)- amino)-1,5-naphthyridin-3-yl)ethanone;
2-chloro-4-(8-((4-(dimethylamino)cyclohexyl)amino)-7-(methylsulfonyl)-1,5-naphthyridin-2-yl)-6-fluorophenol;
1-(6-(3,5-dichloro-4-hydroxyphenyl)-4-((1-(1-methylpiperidin-4-yl)-1H-pyrazol-4-yl)- amino)-1,5-naphthyridin-3-yl)ethanone;
1-(6-(3,5-dichloro-4-hydroxyphenyl)-4-(4-((4-methylpiperazin-1-yl)methyl)-phenylamino)-1,5-naphthyridin-3-yl)ethanone;
1-(6-(3-chloro-5-fluoro-4-hydroxyphenyl)-4-(4-((4-methylpiperazin-1-yl)methyl)-phenylamino)-1,5-naphthyridin-3-yl)ethanone;
1-(6-(3,5-dichloro-4-hydroxyphenyl)-4-(4-(2-(pyrrolidin-1-yl)ethyl)piperidin-1-yl)-1,5-naphthyridin-3-yl)ethanone;
1-(6-(3-chloro-5-fluoro-4-hydroxyphenyl)-4-(4-(2-(pyrrolidin-1-yl)ethyl)piperidin-1-yl)-1,5-naphthyridin-3-yl)ethanone;
1-(6-(3,5-dichloro-4-hydroxyphenyl)-4-(6-(2-(dimethylamino)ethylamino)pyridin-3- ylamino)-1,5-naphthyridin-3-yl)ethanone;
1-(6-(3-chloro-5-fluoro-4-hydroxyphenyl)-4-(6-(2-(dimethylamino)ethylamino)-pyridin-3-ylamino)-1,5-naphthyridin-3-yl)ethanone;
(S)-(4-(6-(3-aminopiperidin-1-yl)pyridin-3-ylamino)-6-(3, 5-dichloro-4-hydroxyphenyl)-1,5-naphthyridin-3-yl)(cyclopropyl)methanone;
1-(4-((2-(3-aminopyrrolidin-1-yl)pyrimidin-5-yl)amino)-6-(3,5-dichloro-4-hydroxyphenyl)-1,5-naphthyridin-3 -yl)ethanone;
1-(4-(4-((dimethylamino)methyl)cyclohexylamino)-6-(1H-pyrazol-4-yl)-1,5-naphthyridin-3-yl)ethanone;
1-(6-(3,5-dichloro-4-hydroxyphenyl)-4-(4-(hydroxymethyl)cyclohexylamino)-1,5-naphthyridin-3-yl)ethanone;
1-[6-(3,5-dichloro-4-hydroxyphenyl)-4-{4-[(dimethylamino)methyl]-cyclohexylamino}-1,5-naphthyridin-3-yl]-2-hydroxyethanone;
1-(6-(3,5-dichloro-4-hydroxyphenyl)-4-(1-methylpiperidin-4-ylamino)-1,5-naphthyridin-3-yl)ethanone;
1-(6-(3-chloro-5-fluoro-4-hydroxyphenyl)-4-(1-methylpiperidin-4-ylamino)-1,5-naphthyridin-3-yl)ethanone;
1-{6-[3,5-dichloro-4-hydroxyphenyl]-4-[4-(morpholinomethyl)cyclohexylamino]-1,5-naphthyridin-3-yl}ethanone;
1-(6-(3,5-dichloro-4-hydroxyphenyl)-4-(4-(((2-hydroxyethyl)(methyl)amino)methyl)- cyclohexylamino)-1,5-naphthyridin-3-yl)ethanone;
1-(6-(3-chloro-5-fluoro-4-hydroxyphenyl)-4-(4-(((2-hydroxyethyl)(methyl)amino)-methyl)cyclohexylamino)-1,5-naphthyridin-3-yl)ethanone;
1-(6-(3,5-difluoro-4-hydroxyphenyl)-4-(4-((dimethylamino)methyl)cyclohexylamino)- 1,5-naphthyridin-3-yl)ethanone;
1-(6-(3,5-dichloro-4-hydroxyphenyl)-4-((6-(3-(dimethylamino)pyrrolidin-1-yl)pyridin-3-yl)amino)-1,5-naphthyridin-3-yl)ethanone;
1-(6-(3-chloro-5-fluoro-4-hydroxyphenyl)-4-((6-(3-(dimethylamino)pyrrolidin-1-yl)-pyridin-3-yl)amino)-1,5-naphthyridin-3-yl)ethanone;
1-(6-(3,5-dichloro-4-hydroxyphenyl)-4-(6-(3-(methylamino)pyrrolidin-1-yl)pyridin-3-ylamino)-1,5-naphthyridin-3-yl)ethanone;
1-(6-(3 -chloro-5 -fluoro-4-hydroxyphenyl)-4-(6-(3 -(methylamino)pyrrolidin-1-yl)-pyridin-3-ylamino)-1,5-naphthyridin-3-yl)ethanone;
1-(6-(1H-benzo[d]imidazol-5-yl)-4-(4-((dimethylamino)methyl)cyclohexylamino)-1,5- naphthyridin-3-yl)ethanone;
1-(4-((4-((dimethylamino)methyl)cyclohexylamino)-6-(pyridin-4-yl)-1,5-naphthyridin-3-yl)ethanone;
5-(7-acetyl-8-(4-((dimethylamino)methyl)cyclohexylamino)-1,5-naphthyridin-2-yl)- pyrimidine-2-carbonitrile;
1-(6-(3,5-dimethyl-1H-pyrazol-4-yl)-4-(4-((dimethylamino)methyl)cyclohexylamino)-1,5-naphthyridin-3-yl)ethanone;
1-(4-(4-((dimethylamino)methyl)cyclohexylamino)-6-(4-hydroxy-3,5-dimethylphenyl)-1,5-naphthyridin-3-yl)ethanone;
1-(6-(3,5-dichloro-4-hydroxyphenyl)-4-(4-(pyrrolidin-1-ylmethyl)phenylamino)-1,5- naphthyridin-3-yl)ethanone;
1-(6-(3,5-dichloro-4-hydroxyphenyl)-4-(4-(pyrrolidin-1-ylmethyl)cyclohexylamino)- 1,5-naphthyridin-3-yl)ethanone;
1-(6-(3-chloro-5-fluoro-4-hydroxyphenyl)-4-(4-(pyrrolidin-1-ylmethyl)cyclohexyl-amino)-1,5-naphthyridin-3-yl)ethanone;
1-(6-(3,5-dichloro-4-hydroxyphenyl)-4-(4-((4-methylpiperazin-1-yl)methyl)cyclo-hexylamino)-1,5-naphthyridin-3-yl)ethanone;
1-(4-(6-(3-aminopiperidin-1-yl)pyridin-3-ylamino)-6-(3,5-dichloro-4-hydroxyphenyl)-1,5-naphthyridin-3-yl)ethanone;
1-(4-(6-(3-aminopiperidin-1-yl)pyridin-3-ylamino)-6-(3-chloro-5-fluoro-4-hydroxy-phenyl)-1, 5-naphthyridin-3-yl)ethanone;
1-(4-(4-aminocyclohexylamino)-6-(3,5-dichloro-4-hydroxyphenyl)-1,5-naphthyridin-3-yl)ethanone;
1-[4-(4-aminocyclohexylamino)-6-(3-chloro-5-fluoro-4-hydroxyphenyl)-1,5-naphthyridin-3-yl]ethanone;
1-(6-(3-chloro-5-fluoro-4-hydroxyphenyl)-4-(4-((4-methylpiperazin-1-yl)methyl)-cyclohexylamino)-1,5-naphthyridin-3-yl)ethanone;
N-(4-(3-acetyl-6-(3-chloro-5-fluoro-4-hydroxyphenyl)-1,5-naphthyridin-4-ylamino)- cyclohexyl)-2-amino-3-methylbutanamide;
1-(6-(3,5-dichloro-4-hydroxyphenyl)-4-(4-(piperazin-1-ylmethyl)cyclohexylamino)-1,5-naphthyridin-3-yl)ethanone;
(S)-1-(4-(6-(3-aminopiperidin-1-yl)pyridin-3-ylamino)-6-(3,5-dichloro-4-hydroxyphenyl)-1,5-naphthyridin-3 -yl)ethanone;
(S)-1-(4-(6-(3-aminopiperidin-1-yl)pyridin-3-ylamino)-6-(3-chloro-5-fluoro-4-hydroxy-phenyl)-1,5-naphthyridin-3-yl)ethanone;
N-(4-((3-acetyl-6-(3,5-dichloro-4-hydroxyphenyl)-1,5-naphthyridin-4-yl)amino)cyclo-hexyl)-2-aminopropanamide;
N-(4-(3-acetyl-6-(3-chloro-5-fluoro-4-hydroxyphenyl)-1,5-naphthyridin-4-ylamino)-cyclohexyl)-2-aminopropanamide;
(S)-N-((1R,4S)-4-(3-acetyl-6-(3,5-dichloro-4-hydroxyphenyl)-1,5-naphthyridin-4-yl-amino)cyclohexyl)pyrrolidine-2-carboxamide;
(S)-N-((1R,4S)-4-(3-acetyl-6-(3-chloro-5-fluoro-4-hydroxyphenyl)-1,5-naphthyridin-4-ylamino) cyclohexyl)pyrrolidine-2-carboxamide;
1-(6-(3-hydroxypyrrolidin-1-yl)-4-(4-((3-hydroxypyrrolidin-1-yl)methyl)cyclohexyl- amino)-1,5-naphthyridin-3-yl)ethanone;
1-(6-(pyrrolidin-1-yl)-4-(4-(pyrrolidin-1-ylmethyl)cyclohexylamino)-1,5-naphthyridin-3-yl)ethanone;
N-(4-(3-acetyl-6-(3,5-dichloro-4-hydroxy phenyl)-1,5-naphthyridin-4-ylamino)-cyclohexyl)-2-amino-3-methylbutanamide;
[6-(3-chloro-5-fluoro-4-hydroxyphenyl)-4-[4-(dimethylamino)cyclohexylamino]-1,5-naphthyridin-3-yl](cyclopropyl)methanone;
cyclopropyl[6-(3,5-dichloro-4-hydroxyphenyl)-4-[4-(dimethylamino)cyclohexyl-amino]-1,5-naphthyridin-3-yl]methanone;
1-(4-{4-[(dimethylamino)methyl]cyclohexylamino}-6-(1H-pyrrolo[2,3-b]pyridin-5-yl)-1,5-naphthyridin-3-yl)ethanone;
(S)-{4-[6-(3-aminopiperidin-1-yl)pyridin-3-ylamino]-6-(3-chloro-5-fluoro-4-hydroxyphenyl)-1,5-naphthyridin-3-yl}(cyclopropyl)methanone;
1-(4-{4-[(dimethylamino)methyl]cyclohexylamino}-6-(4-methoxyphenyl)-1,5-naphthyridin-3-yl)ethanone;
1-[6-(3,5-dichloro-4-methoxyphenyl)-4-{4-[(dimethylamino)methyl]cyclohexyl-amino}-1,5-naphthyridin-3-yl]ethanone;
1-(4-{4-[(dimethylamino)methyl]cyclohexylamino}-6-(6-hydroxypyridin-3-yl)-1,5-naphthyridin-3-yl)ethanone;
5-(7-acetyl-8-{4-[(dimethylamino)methyl]cyclohexylamino}-1,5-naphthyridin-2-yl)picolinonitrile;
1-(4-{4-[(dimethylamino)methyl]cyclohexylamino}-6-(4-hydroxyphenyl)-1,5-naphthyridin-3-yl)ethanone;
1-[6-(3,5-dichloro-4-hydroxyphenyl)-4-{[4-(dimethylamino)cyclohexyl]methyl-amino}-1,5-naphthyridin-3-yl)ethanone;
1-[6-(3-chloro-5-fluoro-4-hydroxyphenyl)-4-{[4-(dimethylamino)cyclohexyl]-methylamino}-1,5-naphthyridin-3-yl]ethanone;
1-[6-(3-chloro-5-fluoro-4-hydroxyphenyl)-4-(4-hydroxycyclohexylamino)-1,5-naphthyridin-3-yl] ethanone;
1-[6-(3,5-dichloro-4-hydroxyphenyl)-4-(4-hydroxycyclohexylamino)-1,5-naphthyridin-3-yl]ethanone;
1-[6-(3-chloro-5-fluoro-4-hydroxyphenyl)-4-{cis-4-[(dimethylamino)methyl]cyclohexylamino}-1,5-naphthyridin-3-yl]ethanone;
1-[6-(3,5-dichloro-4-hydroxyphenyl)-4-{cis-4-[(dimethylamino)methyl]cyclohexylamino}-1,5-naphthyridin-3-yl]ethanone;
(R)-1-{4-[6-(3-aminopiperidin-1-yl)pyridin-3-ylamino]-6-(3,5-dichloro-4-hydroxyphenyl)-1,5-naphthyridin-3-yl}ethanone;
(R)-1-{4-[6-(3-aminopiperidin-1-yl)pyridin-3-ylamino]-6-(3-chloro-5-fluoro-4-hydroxyphenyl)-1,5-naphthyridin-3-yl}ethanone; and
pharmaceutically acceptable salts thereof.

12. The compound or a pharmaceutically acceptable salt thereof according to clause 1, which is selected from the group consisting of the following compounds:
1-(6-(3,5-dichloro-4-hydroxyphenyl)-4-(trans-(4-(dimethylamino)cyclohexyl)amino)-1,5-naphthyridin-3-yl)ethanone;
Cyclopropyl (6-(3,5-dichloro-4-hydroxyphenyl)-4-(trans-4-((dimethylamino)methyl)-cyclohexylaminoamino)-1,5-naphthyridin-3 -yl) methanone;
(6-(3-chloro-5-fluoro-4-hydroxyphenyl)-4-(trans-4-((dimethylamino)methyl)-cyclohexylamino)-1,5-naphthyridin-3-yl) (cyclopropyl) methanone;
1-(6-(3,5-dichloro-4-hydroxyphenyl)-4-((trans-4-((dimethylamino)methyl)cyclohexyl)-amino)-1,5-naphthyridin-3-yl)ethanone;
1-(6-(3-chloro-5-fluoro-4-hydroxyphenyl)-4-((trans-4-((dimethylamino)methyl)-cyclohexyl)amino)-1,5-naphthyridin-3-yl) ethanone;
1-(6-(3,5-dichloro-4-hydroxyphenyl)-4-((trans-4-(2-(dimethylamino)ethyl)cyclohexyl)-amino)-1,5-naphthyridin-3-yl)ethanone;
(S)-(4-(6-(3 -aminopiperidin-1 -yl)pyridin-3 -ylamino)-6-(3, 5-dichloro-4-hydroxyphenyl)-1,5-naphthyridin-3-yl)(cyclopropyl)methanone;
1-[6-(3,5-dichloro-4-hydroxyphenyl)-4-{trans-4-[(dimethylamino)methyl]cyclohexylamino}-1,5-naphthyridin-3-yl]-2-hydroxyethanone;
1-(4-(6-(3-aminopiperidin-1-yl)pyridin-3-ylamino)-6-(3,5-dichloro-4-hydroxyphenyl)-1,5-naphthyridin-3-yl)ethanone;
1-(4-(6-(3-aminopiperidin-1-yl)pyridin-3-ylamino)-6-(3 -chloro-5 -fluoro-4-hydroxy- phenyl)-1,5-naphthyridin-3-yl)ethanone;
(S)-1-(4-(6-(3-aminopiperidin-1-yl)pyridin-3-ylamino)-6-(3,5-dichloro-4-hydroxyphenyl)-1,5-naphthyridin-3 -yl)ethanone;
(S)-1-(4-(6-(3 -aminopiperidin-1 -yl)pyridin-3 -ylamino)-6-(3 -chloro-5-fluoro-4-hydroxy-phenyl)-1,5-naphthyridin-3-yl)ethanone;
(S)-{4-[6-(3-aminopiperidin-1-yl)pyridin-3-ylamino]-6-(3-chloro-5-fluoro-4-hydroxy-phenyl)-1,5-naphthyridin-3-yl} (cyclopropyl) methanone;
(R)-1-{4-[6-(3-aminopiperidin-1-yl)pyridin-3-ylamino]-6-(3,5-dichloro-4-hydroxyphenyl)-1,5-naphthyridin-3-yl}ethanone;
(R)-1-{4-[6-(3-aminopiperidin-1-yl)pyridin-3-ylamino]-6-(3-chloro-5-fluoro-4-hydroxyphenyl)-1,5-naphthyridin-3-yl}ethanone;
(R)-(4-{[6-(3-aminopiperidin-1-yl)pyridin-3-yl]amino}-6-(3,5-dichloro-4-hydroxyphenyl)-1,5-naphthyridin-3-yl) (cyclopropyl)methanone;
(R)-(4- { {[6-(3 -aminopiperidin-1 -yl)pyridin-3-yl]amino}-6-(3 -chloro-5-fluoro-4-hydroxyphenyl)-1,5-naphthyridin-3-yl)(cyclopropyl)methanone;
1-[6-(3,5-dichloro-4-hydroxyphenyl)-4- { [trans-4-(dimethylamino)cyclohexyl] amino}-1,5-naphthyridin-3-yl)-2-hydroxyethanone dihydrochloride;
1-[6-(3-chloro-5-fluoro-4-hydroxyphenyl)-4-({trans-4-[(dimethylamino)methyl]cyclohexyl}amino)-1,5-naphthyridin-3-yl)]-2-hydroxyethanone dihydrochloride;
1-[6-(3-chloro-5-fluoro-4-hydroxyphenyl)-4-({trans-4-[(dimethylamino)methyl] cyclohexyl}amino)-1,5-naphthyridin-3-yl)]propan-1-one dihydrochloride;
1-[6-(3,5-dichloro-4-hydroxyphenyl)-4-({trans-4-[(dimethylamino)methyl] cyclohexyl}amino)-1,5-naphthyridin-3-yl)]propan-1-one dihydrochloride;
(S)-1-(4-{[6-(3-aminopiperidin-1-yl)pyridin-3-yl]amino}-6-(3,5-dichloro-4-hydroxyphenyl)-1,5-naphthyridin-3-yl)propan-1-one trihydrochloride;
(S)-1-(4{[6-(3-aminopiperidin-1-yl)pyridin-3-yl]amino}-6-(3-chloro-5-fluoro-4-hydroxyphenyl)-1,5-naphthyridin-3-yl)propan-1-one trihydrochloride;
1-[6-(3,5-dichloro-4-hydroxyphenyl)-4-({4-[((R)-3-fluoropyrrolidin-lyl)methyl] cyclohexyl}amino)-1,5-naphthyridin-3-yl]ethanone dihydrochloride;
(S)-(4-((6-(3-aminopiperidin-1-yl)pyridin-3-yl)amino)-6-(3-chloro-5-fluoro-4-hydroxyphenyl)-1,5-naphthyridin-3-yl)(cyclobutyl)methanone dihydrochloride;
(6-(3,5-dichloro-4-hydroxyphenyl)-4-((4-[(dimethylamino)methyl{cyclohexyl) amino)-1,5-naphthyridin-3-yl)(cyclobutyl)methanone dihydrochloride;
(6-(3-chloro-5-fluoro-4-hydroxyphenyl)-4-((4-((dimethylamino)methyl)cyclohexyl) amino)-1,5-naphthyridin-3-yl)(cyclobutyl)methanone dihydrochloride;
(S)-(4- {[6-(3 -aminopiperidin-1 -yl)pyridin-3 -yl]amino } -6-(3 -chloro-5-fluoro-4-hydroxyphenyl)-1,5-naphthyridin-3-yl)(cyclobutyl)methanone;
(R)-1-(4-((6-(3 -aminopiperidin-1-yl)pyridin-3 -yl)amino)-6-(3,5-dichloro-4-hydroxyphenyl)-1,5-naphthyridin-3-yl)propan-1-one trihydrochloride;
(R)-1-(4- {[6-(3-aminopiperidin-1 -yl)pyridin-3-yl]amino}-6-(3,5-dichloro-4-hydroxyphenyl)-1,5-naphthyridin-3-yl)-2-methylpropan-1-one trihydrochloride;
1-[6-(3,5-dichloro-5-4-hydroxyphenyl)-4-({trans-4-[(dimethylamino)methyl]cyclohexyl} amino)-1,5-naphthyridin-3-yl]-2-methylpropan-1-one dihydrochloride;
1-[6-chloro-4-({trans-4-[(dimethylamino)methyl]cyclohexyl}amino)-1,5-naphthyridin-3-yl]-2-methylpropan-1-one dihydrochloride; and
pharmaceutically acceptable salts thereof.

Optionally, an N to C substitution is applied at the center of the molecule according to any of the clauses above.

1-26. (canceled)

27. An inhibitor of maternal embryonic leucine zipper kinase (“MELK”), receptor-related 2 (“ROR2”), and/or MELK and ROR2.

28. A method of treating a subject diagnosed with brain tumor, glioma, a brainstem glioma, or glioblastoma multiforme diffuse intrinsic pontine glioma (“DIPG”), the method comprising:
administering the inhibitor of claim 27 to the subject.

29. The method according to claim 28, wherein the subject is diagnosed with DIPG characterized by an overexpression of MELK and/or an overexpression of ROR2.

30. The inhibitor of claim 27, wherein the inhibitor is an inhibitor of MELK, and has an IC50 selected from the group consisting of <0.1 µM, <50 nM, and <1 nM with respect to inhibiting MELK.

31. The inhibitor of claim 27, wherein the inhibitor is an inhibitor of ROR2, and has an IC50 selected from the group consisting of <0.1 µM, <50 nM, and <1 nM with respect to inhibiting ROR2.

32. The inhibitor of claim 27, wherein the inhibitor has the following structure: 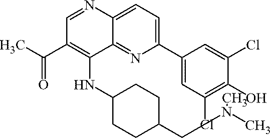 [SMILES: CC(=O)c1cnc2ccc(-c3cc(Cl)c(O)c(Cl)c3)nc2c1NC1CCC(CN(C)C)CC1]
or a solvate or pharmaceutically acceptable salt thereof.

33. The inhibitor of claim 27, wherein the inhibitor has the following structure: 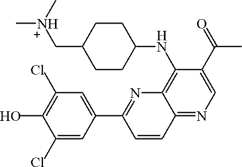 [SMILES: CC(=O)c1cnc2ccc(-c3cc(Cl)c(O)c(Cl)c3)nc2c1NC1CCC(C[NH+](C)C)CC1]
or a solvate or pharmaceutically acceptable salt thereof.

34. The inhibitor of claim 27, wherein the inhibitor has the following structure: 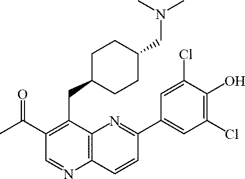 [SMILES: CC(=O)c1cnc2ccc(-c3cc(Cl)c(O)c(Cl)c3)nc2c1C[C@H]1CC[C@H](CN(C)C)CC1]
or a solvate or pharmaceutically acceptable salt thereof.

35. The inhibitor of claim 27, wherein the inhibitor has the formula CC(O)=c3cnc2ccc(c1cc(Cl)c(O)c(Cl)c1)[nH]c2c3CC4CCC(CN(C)C)CC4, or a solvate or pharmaceutically acceptable salt thereof.

36. A composition comprising:
the inhibitor of claim 27, and
a compound selected from the group consisting of (2R,3R,4S,5R)-2-(6-amino-9H-purin-9-yl)-5-(hydroxymethyl)tetrahydro-3,4-furandiol, a P-glycoprotein inhibitor, 1-[6-amino-9-[(2R,3R,4S,5R)-3,4-dihydroxy-5-(hydroxymethyl)oxolan-2-yl]purin-2-yl]- N-methylpyrazole-4-carboxamide, an Abcb1a inhibitor, an Abcb1b inhibitor, N-[3-(4-Morpholinyl)propyl]-5,7-diphenylpyrazolo[1,5-a]pyrimidine-3-carboxamide, mannitol, an Abcg2 inhibitor, N-[4-[2-(3,4-dihydro-6,7-dimethoxy-2(1H)-isoquinolinyl)ethyl]phenyl]-9,10-dihydro-5-methoxy-9-oxo-4-acridinecarboxamide, and a combination of any thereof.

37. The method according to claim 28, further comprising:
utilizing convection enhanced delivery to administer the inhibitor, and/or
wherein at least one catheter is applied for delivery of the inhibitor in tumor tissue of the subj ect.

38. The method according to claim 28, further comprising:
utilizing ultrasound to disrupt the subject’s blood-brain barrier, or
by localized exposure to high-intensity focused ultrasound disrupting the local blood-brain barrier of tumor tissue with a frequency range 500 kHz - 1.5 MHz.

39. The method according to claim 28,
wherein delivery across the subject’s blood-brain barrier utilizes encapsulation of the inhibitor in a liposome, or
wherein delivery across the subject’s blood-brain barrier utilizes encapsulation of the inhibitor in a liposome, and the liposome has molecules on its surface that are actively transported over the blood-brain barrier, wherein the molecules bind a endothelial cell receptor or the endothelial cell receptor for transferrin or insulin.

40. The method according to claim 28, wherein the subject has a brain tumor that is a brain metastasis, astrocytoma, glioblastoma, oligodendroglioma, ependymomas, optic nerve glioma, and/or a mixed glioma.

41. The method according to claim 28, wherein administration of the inhibitor is orally, nasally, or intravenously.

42. The method according to claim 28, wherein the inhibitor is administered in to the subject in an amount between 0.001 mg/kg per day and 50 mg/kg per day.

43. The method according to claim 28, wherein the inhibitor is administered in to the subject in an amount between 10 mg/m² per day and 2000 mg/m² per day.

44. The method according to claim 28, wherein the inhibitor and at least one unit or of β-(1→4)-linked D-glucosamine and/or at least one unit of N-acetyl-D-glucosamine is administered.

45. The method according to claim 44, which is administered to the subject’s nasal olfactory region.

46. A method of treating a subject diagnosed with a brain tumor, glioma, a brainstem glioma, or glioblastoma multiforme diffuse intrinsic pontine glioma (DIPG), the method comprising:
inhibiting maternal embryonic leucine zipper kinase (“MELK”) and/or receptor-related 2 (“ROR2”) in the subject.
